(19) United States
(12) Patent Application Publication
(10) Pub. No.: US 2017/0351768 A1
(43) Pub. Date: Dec. 7, 2017

(54) SYSTEMS AND METHODS FOR CONTENT TARGETING USING EMOTIONAL CONTEXT INFORMATION

(71) Applicant: Intertrust Technologies Corporation, Sunnyvale, CA (US)

(72) Inventor: Yutaka Nagao, Cupertino, CA (US)

(73) Assignee: Intertrust Technologies Corporation, Sunnyvale, CA (US)

(21) Appl. No.: 15/611,509

(22) Filed: Jun. 1, 2017

Related U.S. Application Data
(60) Provisional application No. 62/345,384, filed on Jun. 3, 2016.

(51) Int. Cl. G06F 17/30; A61B 5/00; A61B 5/16; A61B 7/04; G06Q 30/02; A61B 5/0205; A61B 5/021; A61B 5/024

(52) U.S. Cl. CPC G06F 17/30867; G06Q 30/0269; A61B 5/4266; A61B 5/02055; A61B 5/165; A61B 5/0077; A61B 7/04; A61B 5/024; A61B 5/021; A61B 2503/12; A61B 2562/0247

(57) ABSTRACT

This disclosure relates to systems and methods that use contextual information relating to a user's emotions and/or moods in connection with information targeting and content tagging. In some embodiments, user moods and/or emotional states may be determined and/or otherwise inferred using certain contextual information collected using one or more sensors included in devices associated with a user. Obtaining information relating to a user's mood and/or emotional state may allow for, among other things, more efficient targeting of content, search results, and/or other information that is well matched to a user's interests at a given point in time. In further embodiments, information relating to a user's mood and/or emotional state may be used to tag and/or otherwise associate content with information relating to the user's mood and/or emotional state while viewing and/or capturing the content.

### RELATED APPLICATIONS

This application claims the benefit of priority under 35 U.S.C. §119(e) to U.S. Provisional Application No. 62/345,384, filed Jun. 3, 2016, and entitled “SYSTEMS AND METHODS FOR CONTENT TARGETING USING EMOTIONAL CONTEXT INFORMATION,” which is hereby incorporated by reference in its entirety.

### COPYRIGHT AUTHORIZATION

Portions of the disclosure of this patent document may contain material which is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### SUMMARY

The present disclosure relates generally to information targeting and content tagging services. More specifically, but not exclusively, the present disclosure relates to using contextual information relating to a user's emotions in connection with information targeting and content tagging operations.

As users interact with personal electronic devices and services, including mobile electronic devices, the Internet and other connected services, a variety of information may be collected. For example, device usage information may be obtained from personal electronic devices indicative of a user's interaction with the devices and/or various applications or features executing thereon. In addition, a variety of environmental information may be obtained from sensors included in a personal electronic device indicative of user and/or device location, motion, and/or other activities.

Systems and methods disclosed herein relate to determining various information associated with a user including, for example, user interests, moods, and/or emotional states, based on information obtained from personal electronic devices associated with the user. Consistent with embodiments disclosed herein, user moods and/or emotional states may be determined and/or otherwise inferred using certain contextual information collected using one or more sensors included in devices associated with a user. Such contextual information may include, without limitation, audio information relating to a user's voice, image or video information relating to a user's facial expressions, biometric information relating to a variety of user philological responses (e.g., heart rate, blood pressure, perspiration, etc.), and/or the like.

Obtaining information relating to a user's mood and/or emotional state may allow for, among other things, more efficient targeting of content, search results, and/or other information that is well matched to a user's interests at a given point in time. In further embodiments, information relating to a user's mood and/or emotional state may be used to tag and/or otherwise associate content with information relating to the user's mood and/or emotional state while viewing and/or capturing the content.

### BRIEF DESCRIPTION OF THE DRAWINGS

The inventive body of work will be readily understood by referring to the following detailed description in conjunction with the accompanying drawings, in which:

FIG. 1 illustrates information targeting using emotional context information consistent with embodiments of the present disclosure.

FIG. 2 illustrates exemplary emotional context information and associated emotional states consistent with embodiments disclosed herein.

FIG. 3 illustrates an exemplary device architecture for associating content with emotional context information consistent with embodiments disclosed herein.

FIG. 4 illustrates an exemplary method for associating contextual information with content consistent with embodiments of the present disclosure.

FIG. 5 illustrates an exemplary method for emotional response targeting using contextual information consistent with embodiments of the present disclosure.

FIG. 6 illustrates an exemplary system that may be used to implement embodiments of the systems and methods of the present disclosure.

### DETAILED DESCRIPTION

A detailed description of the systems and methods consistent with embodiments of the present disclosure is provided below. While several embodiments are described, it should be understood that the disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

The embodiments of the disclosure may be understood by reference to the drawings, wherein like parts may be designated by like numerals or descriptions. The components of the disclosed embodiments, as generally described and illustrated in the figures herein, could be arranged and designed in a wide variety of different configurations. Thus, the following detailed description of the embodiments of the systems and methods of the disclosure is not intended to limit the scope of the disclosure but is merely representative of possible embodiments of the disclosure. In addition, the steps of any method disclosed herein do not necessarily need to be executed in any specific order, or even sequentially, nor need the steps be executed only once, unless otherwise specified.

Consistent with embodiments disclosed herein, personal information relating to a user may be obtained and used in connection with efficiently targeting information to a particular user and/or content tagging operations. Such personal information may include, without limitation, usage information relating to usage devices associated with a user, environmental information obtained from sensors included in such devices, user volunteered personal information, and/or a measure of the user's emotional state and/or mood obtained based on associated contextual information. Based on such information, a user's interests at a given time may be inferred, and matched content, search results, and/or other information may be targeted for delivery to the user. Similarly, content generated, and/or viewed by a user may be tagged with personal information associated with a user including, without limitation, information relating to an emotional state and/or mood of a user at the time they viewed and/or captured the content.

Embodiments disclosed herein may be used in connection with a variety of information targeting systems and methods. For example, the systems and methods described herein can, for example, be used in connection with advertisement matching and/or advertisement targeting technologies such as those described in commonly assigned co-pending U.S. patent application Ser. No. 12/785,406, filed May 21, 2010 (“the '406 application”), which is incorporated herein by reference in its entirety.

Personal information related to a user may be obtained in a variety of ways, including through monitoring user interactions with devices and services. For example, demographic information about the user (e.g., age, gender, etc.), usage history and preferences of the user, information about the user's device, content preference information (e.g., preferred genres, artists, etc.), information about the user or the user's environment (e.g., time of day, global positioning system (“GPS”) coordinates, etc.), and/or any other available information relating to a user and/or an associated device may be obtained. In some circumstances, this personal information may be volunteered directly by a user. For example, when registering a device, a user may voluntary provide personal demographic information to a device manufacturer and/or service provider.

Consistent with embodiments disclosed herein, personal information related to a user may further comprise contextual information indicative of a user's mood and/or emotional state, generally referred to herein as emotional context information and/or contextual information. For example, information relating to a user's voice (e.g., a tone, decibel level, content of speech, and/or the like) may be used to infer a mood and/or emotional state associated with a user (e.g., happy, sad, angry, hungry, excited, etc.). Image and/or video information relating to a user's facial expressions may also be used to infer an associated mood and/or emotional state of the user. In yet further embodiments, information relating to certain physiological responses of a user including, for example, heart rate, blood pressure, temperature, perspiration, and/or the like (e.g., obtained using wearable health monitoring devices and/or the like), may also be used to infer an associated mood and/or emotional state of the user.

As discussed above, personal information provided by a user and/or generated based on a user's activities may, among other things, be utilized to effectively match information including, without limitation, advertisements, content, and/or search results, to the interests of the user. This may be achieved utilizing, for example, the ad-matching technologies described in the '406 application. In certain embodiments, information matching may be performed locally on a user's device. Alternatively, information—matching may performed by a trusted third party and/or a content or search service. Further, in circumstances where a user utilizes multiple devices and/or services to consume content, personal information may be managed, shared, and/or aggregated between the devices and/or services to generate a more detailed and accurate profile of the user's interests. By improving the ability to generate a more detailed profile of a user's interests, managing personal information related to the user between multiple devices can improve information matching services.

In the context of managing, sharing, and aggregating personal information between multiple devices and/or services, the confidentiality of certain private personal information related to the users may be maintained. For example, a user may wish to not have an audio and/or video recording of themselves transferred to third-party systems and/or untrusted services from their local user devices. Accordingly, systems and methods may be deployed that allow for managing the confidentiality of user personal information. In some embodiments, this may be achieved by ensuring that certain personal information is not communicated outside of a user's device, devices, and/or a trusted boundary associated with the user. Additionally, anonymous and/or anonymized versions of personal information may be generated that can be managed, shared, and aggregated between multiple devices and/or services without compromising user privacy. Further, users may specifically restrict access to certain categories and/or types of personal information, while allowing the sharing and aggregating of other types of personal information through one or more articulated policies. Employing such techniques may allow for improved information matching services while maintaining the confidentiality of certain user personal information.

In further embodiments, the disclosed systems and methods may facilitate association of generated and/or rendered content with relevant emotional context information. When content is generated and/or otherwise captured by user device, the generated content may be associated with available emotional context information relating to a user, thereby providing an indication of a user's emotional state and/or mood when the content was generated. For example, a captured photograph may be associated with emotional context information including information relating to a user's voice captured by a microphone and philological information captured by a biometric sensor, thereby providing a way to search and/or otherwise identify a user's emotional state and/or mood when the photograph was taken. In further embodiments, emotional states and/or moods inferred based on the emotional context information may be associated with the generated and/or rendered content.

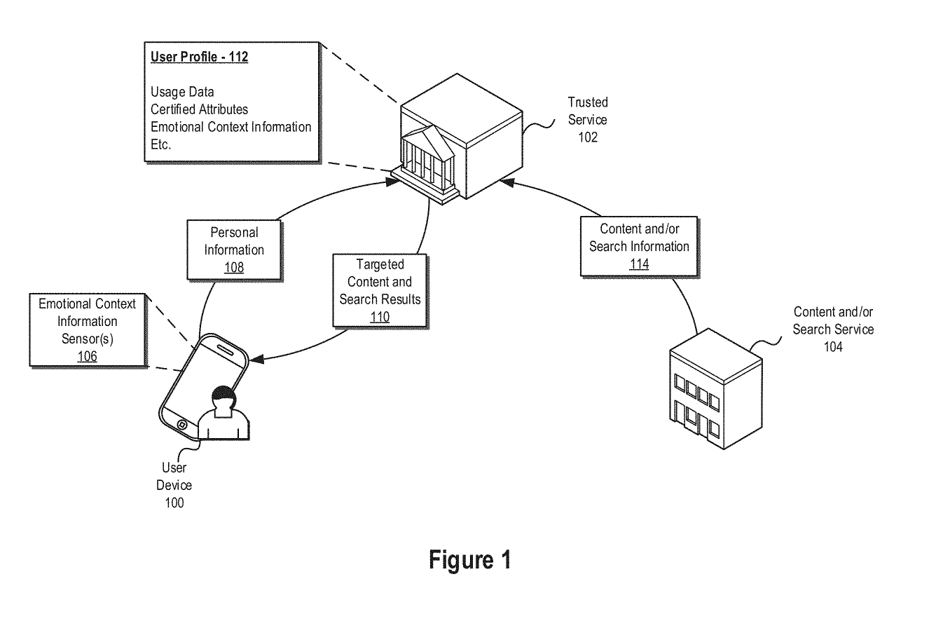

FIG. 1 illustrates information targeting using emotional context information consistent with embodiments of the present disclosure. In certain embodiments, a user device 100 may interact with a trusted service 102 and/or a content/search service 104. Although embodiments disclosed herein are discussed in connection with a trusted service 102 and/or a content/search service 104, it will be appreciated that embodiments may be used in connection with a variety of other suitable information targeting systems.

The user device 100, trusted service 102, content/search service 104, and/or one or more other service providers (not shown) may comprise any suitable computing system or combination of systems configured to implement embodiments of the systems and methods disclosed herein. In certain embodiments, the user device 100, trusted service 102, content/search service 104, and/or other service providers may comprise at least one processor system configured to execute instructions stored on an associated non-transitory computer-readable storage medium. As discussed in more detail below, the user device 100, trusted service 102, content/search service 104, and/or other service providers may further comprise a secure processing unit (“SPU”) configured to perform sensitive operations such as trusted credential and/or key management, secure policy management, and/or other aspects of the systems and methods disclosed herein. The user device 100, trusted service 102, content/search service 104, and/or other service providers may further comprise software and/or hardware configured to enable electronic communication of information between the devices and/or services via one or more associated network connections.

The user device 100, trusted service 102, and/or content/search service 104 may comprise a computing device executing one or more applications configured to implement embodiments of the systems and methods disclosed herein. In certain embodiments, the user device 100 may comprise at least one of a smartphone, a smartwatch, a laptop computer system, a desktop computer system, a gaming system, an entertainment system, a streaming media system, a wearable health monitoring device, a tablet computer, a smart home device, a digital assistant device, a connected appliance, and/or any other computing system and/or device that may be used in connection with the disclosed systems and methods. In certain embodiments, the user device 100 may comprise software and/or hardware (e.g., emotional context information sensors 108) configured to, among other things, obtain personal information 108 including contextual information relating to a user's moods and/or emotional states, infer moods, emotional states, and/or interests of the user based on such personal information 108, and/or match information to the moods, emotional states, and/or interests of the user.

The user device 100, trusted service 102, and/or content/search service 104 may communicate using a network comprising any suitable number of networks and/or network connections. The network connections may comprise a variety of network communication devices and/or channels and may use any suitable communication protocols and/or standards facilitating communication between the connected devices and systems. For example, in some embodiments the network may comprise the Internet, a local area network, a virtual private network, and/or any other communication network utilizing one or more electronic communication technologies and/or standards (e.g., Ethernet and/or the like). In some embodiments, the network connections may comprise a wireless carrier system such as a personal communications system (“PCS”), and/or any other suitable communication system incorporating any suitable communication standards and/or protocols. In further embodiments, the network connections may comprise an analog mobile communications network and/or a digital mobile communications network utilizing, for example, code division multiple access (“CDMA”), Global System for Mobile Communications or Groupe Special Mobile (“GSM”), frequency division multiple access (“FDMA”), and/or time divisional multiple access (“TDMA”) standards. In certain embodiments, the network connections may incorporate one or more satellite communication links. In yet further embodiments, the network connections may use IEEE's 802.11 standards, Bluetooth®, ultra-wide band (“UWB”), Zigbee®, and or any other suitable communication protocol(s).

As discussed above, information services, including search, content, recommendation, and/or advertisement services, may engage in a variety of activities to improve delivery of their services. For example, a search service 104 may use search engine indexing information, ranking information, and cookie information to analyze a user's past search history and to improve future search results. Recommendation and/or advertisement services may use cookies, mobile device sensor information (e.g., installed application information, geolocation information, etc.) to analyze a user to improve targeting of delivered recommendations and/or advertisements. In many instances, information services may use input from a user (e.g., search terms) combined with analytics relating to a user's past behavior.

As a user interacts with a user device 100 (e.g., consumes and/or generates content and/or interacts with applications and/or services) and/or services, the device 100 and/or services may obtain personal information 108 relating to the user. In certain embodiments, this personal information 108 may reflect in part the interests of the user. Personal information 108 may include, among other things, information volunteered by a user (e.g., declared interests) and/or information collected by monitoring a user's activities in connection with an associated device (e.g., device activity information). For example, a user may provide a device 100 with personal identification information (e.g., age, gender, home address, and the like) and/or other preference information (e.g., content preference information including preferred genres, artists, and the like). Similarly, a device 100 may passively collect usage information regarding the types of content a user consumes, the number of times certain content is consumed, application usage information, location-based information relating to a location of the user, and/or the like. Collectively, personal information 108 may include, without limitation, user attributes such as gender, age, content preferences, geographic location, attributes and information associated with a user's friends, contacts, and groups included in a user's social network, and/or information related to content and/or application usage patterns including what content is consumed, content recommendations, advertisement viewing patterns, and the like.

Certain personal information 108 may be volunteered (e.g., provided directly) by a user. For example, when registering or configuring a device 100, a user may voluntarily provide personal demographic information to the device 100, a device manufacturer, and/or a service provider. In certain embodiments, this information may include a user's age, gender, contact information, address, field of employment, and/or the like. User-volunteered personal information may also include content preference information (e.g., preferred genres, preferred artists, etc.). In some embodiments, in lieu of or in addition to collecting personal information as part of a device registration or configuration process, user-volunteered personal information may be provided by a user when registering with a service or at various times during a user's interaction with a device 100 (e.g., concurrent with selection of a particular piece of content, using a particular application, and/or the like).

In further embodiments, personal information 108 may comprise one or more certified attributes acquired from one or more trusted sources that can authenticate certain attributes relating to the user and/or the user device 100 (e.g., attributes relating to age, gender, education, club membership, employer, frequent flyer or frequent buyer status, credit rating, etc.). The user device 100 may also generate and/or collect other attributes from various user events as personal information 108 including, for example, metrics or attributes derivable from a user's history of interactivity with ads, purchasing history, browsing history, content rendering history, application usage history, and/or the like. Further, a variety of environmental attributes may also be included in personal information 108 such as time of day, geographic location, speed of travel, and/or the like.

Personal information 108 may further include information collected by monitoring a user's activities in connection with an associated device 100 and/or services (e.g., device activity information and/or usage data). Usage data may include information regarding the types of content a user consumes, the number of times certain content is consumed, metrics or attributes derived from a user's history of interactions with ads and/or content, information regarding application usage, application usage history, purchasing history, browsing history, content rendering history, and/or the like. In certain embodiments, usage data may be generated locally on a user's device 100 through monitoring of a user's interaction with the device 100. Alternatively or in addition, usage data may be generated by a trusted third party capable of monitoring a user's interaction with a device 100. In some embodiments, usage data may be stored locally on a user's device 100 in a secure manner to protect the integrity of the data and/or be filtered suitably to ensure that it is anonymized in some way before it is transmitted from the device 100.

Consistent with the disclosed embodiments, information services may utilize certain dynamic contextual information or emotional context information in connection with delivery of their services. For example, contextual information indicative of a user's interests, moods, and/or emotional states and a given time, may prove valuable in targeting well-matched information to the user. In some embodiments, such contextual information may be collected using one or more sensors 106 included in devices 100 associated with the user. Device sensors 106 configured to generate contextual information may include, without limitation, one or more video and/or image sensors such as a camera, audio capture sensors such as a microphone, various biometric information configured to capture information relating to one or more physiological responses of a user, light sensors configured to sense an amount of light incident on at least a portion of the user device 100, pressure sensors configured to sense an amount of pressure applied to a touch screen and/or other device interface, and/or the like. In some embodiments, the device sensors 106 may not necessarily be integrated within the device 100, but may be obtained via one or more communicatively-linked associated peripheral devices such as a health monitoring device and/or the like.

Contextual information, including contextual information generated by the emotional context information sensors 106, may comprise, without limitation, audio information relating to a user's voice (e.g., tone, decibel level, ambient background noise, recognized speech content, etc.), image and/or video information relating to a user's facial expressions (e.g., facial expression recognition information, iris dilation, etc.), biometric and/or physiological information (e.g., heart rate, blood pressure, temperature, perspiration, etc.), other sensor data (e.g., interface/display pressure sensor data, light sensor data, etc.), and/or any other relevant information indicative of a user's emotional state and/or mood (e.g., usage of emoji's and/or other ideograms, etc.).

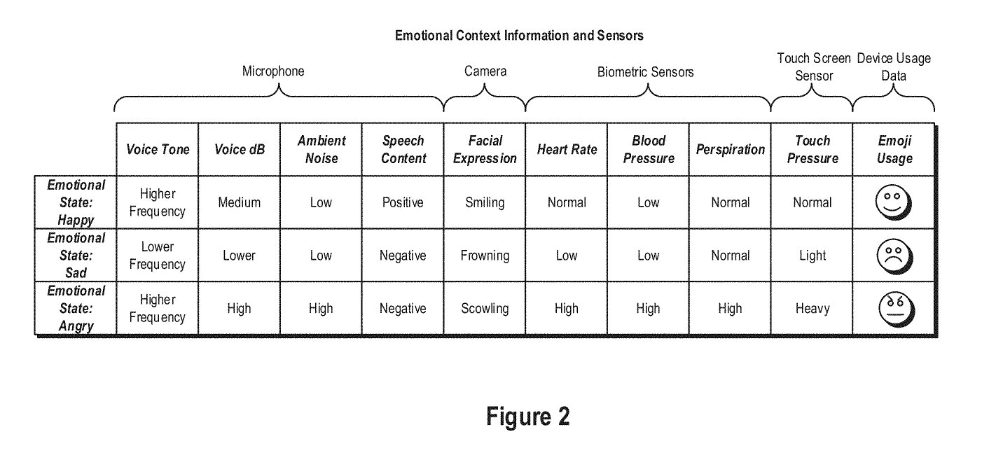

In certain embodiments, contextual information may be used to infer a variety of moods and/or emotional states of a user including, without limitation, anger, contempt, disgust, fear, happiness, indifference, love, sadness, surprise, and/or the like. Inferred moods and/or emotional states may be associated with certain quantified weights and/or metrics indicating a relative likelihood that a user is experiencing a particular mood and/or emotional state based on the associated contextual information. For example, the louder a user's voice, the more likely they are to be feeling anger. FIG. 2 illustrates an example of various moods and/or emotional states and associated contextual information that may be used to infer an associated mood and/or emotional state. It will be appreciated that a variety of other emotional states and/or types of contextual information may be used in connection with the disclosed embodiments, and that any suitable association between contextual information and emotions states may be used. Thus it will be appreciated that the various relationships illustrated in connection with FIG. 2 are presented for explanation purposes, and that other relationships between emotional states and contextual information may be used in connection with the disclosed embodiments.

Referring back to FIG. 1, the user device 100 may transmit personal information 108 relating to the user of the device to a trusted service 102. The trusted service 102 may provide certain functions associated with an information targeting platform such as that described in the '506 application. In some embodiments, based on received personal information 108, the trusted service 102 may populate a profile 112 associated with a user of the user device 100. The profile 112 may comprise a variety of personal information 108 and/or interests, moods, and/or emotional states inferred based on the personal information 108. Information included in the profile 112 may allow information service providers (e.g., a content/search service) to provide to provide a more personalized and targeted experience for users.

In some embodiments, the trusted service 102 may operate in conjunction with the user device 100 and information service providers to provide certain targeted information services. In certain embodiments, the trusted service 102 may function as a trusted intermediary between the user device and an information service, such as the content/search service 104. For example, as illustrated, the trusted service 102 may receive content and/or search information 114 from the content and/or search service. Based on information included in the user profile 112 (e.g., indications of user interests, moods, and/or emotional states) and the received content and/or search information 114, the trusted service 102 may match content and/or search results to a user's interests and transmit matched content and/or search results 110 to the user device.

In one example, a user may be interested in watching a romantic movie, and may use a search service to find a movie title that interests them. The user may provide the keyword “romantic movie,” but such a simplistic search may not necessarily reflect what the user's emotional state was at the time the keyword was provided. If an audible search command for “romantic movie” is provided, however, contextual information that may be derived from the tone of the user's voice may provide information relating to how they felt at the time the search command was issued (e.g., angry, sad, lonely, etc.). Such emotional state information may be weighted and/or otherwise quantified based on analysis of the associated contextual information. For example, one or more scores associated with the derived emotional states (e.g., 0.1 angry, 0.6 sad, 0.3 lonely, etc.) may be generated indicative of a relative likelihood the user is experiencing a particular emotion and/or the relative degree of an experienced emotion.

The search service 104 may have information included in an indexed catalog of romantic movies indicating feedback from others regarding how they felt following watching movies included in the catalog (e.g., marketing research data). In some embodiments, the feedback may comprise weighted, quantified, and/or otherwise scored information relating to emotional states associated with the included movies (e.g., 0.3 angry, 0.4 sad, 0.3 lonely, etc.). Based on a comparison between such indexed information and the emotional state information associated with the user, the search service 104 may return a movie recommendation that best matches the user's emotional state inferred based on the contextual information.

Various aspects of the disclosed embodiments may protect user privacy. For example, if a user is concerned about sending their raw voice recording data to the trusted service 102 and/or the search engine service 104, the raw voice recording data may be analyzed locally on the user's device 100 to determine any associated weighted and/or otherwise parameterized mood and/or emotional state information. This weighted mood and/or emotional state information may then be provided to the trusted service 102 and/or the search engine service 104 instead of the raw voice recording, thereby providing a measure of privacy for the user. In yet further embodiments, certain weighted information may remain local to a user's device 100, and a trusted service 102 and/or information service may send several candidate movie search results to the user device 100 for local matching with the weighted mood and/or emotional state information.

In another example, an advertiser may wish to display a commercial and/or advertisement targeted to a user's interest while they are viewing a program on their television. Image and/or video information obtained by the television and/or another associated device (e.g., a gaming console with a camera and/or the like) may capture a user's face expression at a particular time, which may be used to infer the user's emotional state and/or mood consistent with embodiments disclosed herein. The state and/or mood information may be weighted based on analysis of the associated image and/or video information (e.g., 0.2 hungry, 0.5 happy, 0.3 excited, etc.). Advertisements and/or commercials may be associated with particular emotional states and/or moods by a content provider, and may be matched to particular users based in part on the inferred state and/or mood information.

If a user is concerned about sending raw images or video of their face to the advertising service, the raw images or video may be analyzed locally on the user's device 100 to determine any associated weighted mood and/or emotional state information. This weighted mood and/or emotional state information may then be provided to the advertiser instead of the raw images or video recording, thereby providing a measure of privacy for the user. In yet further embodiments, certain weighted information may remain local to a user's device 100, and an advertiser may send several candidate advertisements to the user device 100 for local matching with the weighted mood and/or emotional state information.

It will be appreciated that a number of variations can be made to the architecture, relationships, and examples presented in connection with FIG. 1 within the scope of the inventive body of work. For example, certain device and/or system functionalities described above may be integrated into a single device and/or system and/or any suitable combination of devices and/or systems in any suitable configuration. Thus it will be appreciated that the architecture, relationships, and examples presented in connection with FIG. 1 are provided for purposes of illustration and explanation, and not limitation.

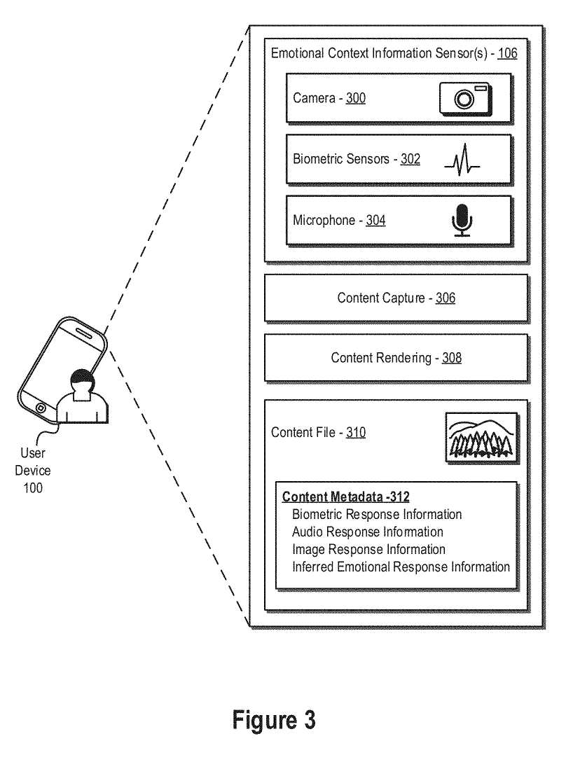

FIG. 3 illustrates an exemplary device architecture for associating content 310 with emotional context information consistent with embodiments disclosed herein. As illustrated, a user device 100 may comprise one or more emotional context information sensors 106 configured to generate and/or otherwise capture contextual information relating to a user's emotional state and/or mood. The emotional context information sensors may include, without limitation, one or more video and/or image sensors such as a camera 300, audio capture sensors such as a microphone 304, various biometric sensors 302 configured to capture information relating to one or more physiological responses of a user, light sensors configured to sense an amount of light incident on at least a portion of the user device 100, pressure sensors configured to sense an amount of pressure applied to a touch screen and/or other device interface, and/or the like. As discussed above, in some embodiments, the device sensors 106 may not necessarily be integral to device 100, but may be associated with one or more peripheral devices in communication with the user device 100, such as a health monitoring device (e.g., a wireless heartrate monitor) and/or the like.

A variety of software modules 306, 308 may be executed by the user device 100 to facilitate user interaction with content 310. In some embodiments, one or more content capture modules 306 may be configured to capture and/or otherwise generate content using the device 100 (e.g., image content 310). For example, the one or more content capture modules 306 may comprise a camera and/or audio recording application confirmed to enable a user to capture video content, image content 310, and/or audio content using the user device. In further embodiments, one or more content rendering modules 308 may be configured to allow a user to view, render, and/or otherwise interact with content using the device 100. For example, a content rendering module 308 may allow a user to view video content, image content 310, and/or playback audio content.

Consistent with embodiments disclosed herein, content 310 generated and/or rendered by user the user device 100 may be associated with contextual information generated by the one or more emotional context information sensors 106. In some embodiments, when content is generated and/or rendered by the user device 100, various contextual information generated by the one or more emotional context information sensors 106 may be included metadata 312 associated with the content 310. For example, as illustrated, when capturing and/or rendering image content, an associated content file 310 may be tagged and/or otherwise associated with metadata 312 that comprises biometric response information, audio response information, image and/or video response information, and/or the like generated by the emotional context information sensors 106 at the time the content was captured and/or rendered by the device 100. In this manner, the metadata 312 information may include information representative of a user's state and/or mood when the content file 310 was captured and/or rendered by the device 100.

In some embodiments, based on analysis of associated contextual information generated by emotional context information sensors 106, emotional states and/or moods associated with the user and/or content file 310 may be inferred. Such inferred emotional response information may further be included in the content metadata 312. As discussed above, inferred emotional response information may comprise weighted, scored, and/or otherwise quantified indications of likely user emotional states and/or moods. In some embodiments, to preserve user privacy, inferred emotional response information may be included in the content metadata 312 without including raw contextual information.

Associating contextual information and/or inferred emotional response information in content metadata may allow for users to interact with generated and/or rendered content files 310 in a variety of ways. In some embodiments, content generated and/or rendered by a device 100 may be searched, indexed, and/or organized, based on the associated emotional state and/or mood experienced by a user when the content was captured and/or rendered. For example, a user may wish to view content that, when viewed previously by the user, caused the user to feel happy. Similarly, a user may wish to view content that, when captured by the user using their device 100, made the user feel euphoric.

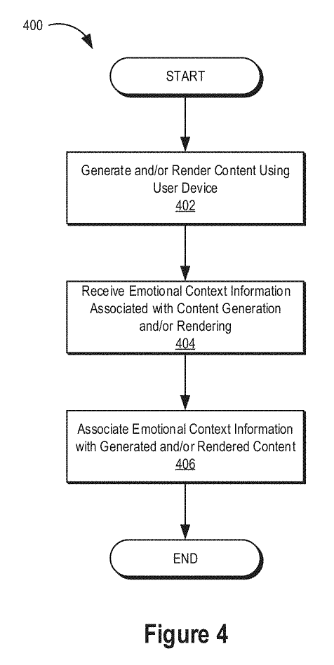

FIG. 4 illustrates an exemplary method 400 for associating contextual information with content consistent with embodiments of the present disclosure. The illustrated method 400 may be implemented in a variety of ways, including using software, firmware, hardware, and/or any combination thereof. In certain embodiments, the method 400 may be implemented by an electronic device associated with a user.

At 402, content may be generated and/or rendered using a user device. For example, image content may be captured by a camera included in and/or otherwise associated with a user device. In another example, video content may be rendered on a user device via an associated display.

Emotional context information may be received from one or more emotional context information sensors included in the user device and/or other devices and/or systems associated with the user device at 404 (e.g., systems in communication with the user device that include emotional content information sensors). As discussed above, the emotional context information may include, without limitation, one or more of audio information relating to a user's voice (e.g., tone, decibel level, ambient background noise, recognized speech content, etc.), image and/or video information relating to a user's facial expressions (e.g., facial expression recognition information, iris dilation, etc.), biometric and/or physiological information (e.g., heart rate, blood pressure, temperature, perspiration, etc.), other sensor data (e.g., pressure sensor data, light sensor data, etc.), and/or any other relevant information indicative of a user's emotional state and/or mood (e.g., usage of emoji's and/or other ideograms, etc.).

In some embodiments, the emotional context information received at 404 may be captured at a time the content was generated and/or rendered at 402 and/or within a certain time period of the content being generated and/or rendered at 402. In this manner, the emotional context information received at 404 may be reflective of a user's emotional state and/or mood when the content was generated and/or rendered.

At 406, the content generated and/or rendered at 402 may be associated with the emotional context information received at 404. For example, the emotional context information received at 404 may be included in metadata associated with the generated and/or rendered content. In some embodiments, based on analysis of the emotional contextual information received at 402, emotional states and/or moods associated with the user may be inferred. Such inferred emotional response information may further be associated with the content generated and/or rendered at 402. As discussed above, inferred emotional response information may comprise weighted, scored, and/or otherwise quantified indications of likely user emotional states and/or moods. In this manner, the content may be searched, indexed, and/or organized based on associated emotional states and/or moods experienced by the user when the content was created and/or rendered.

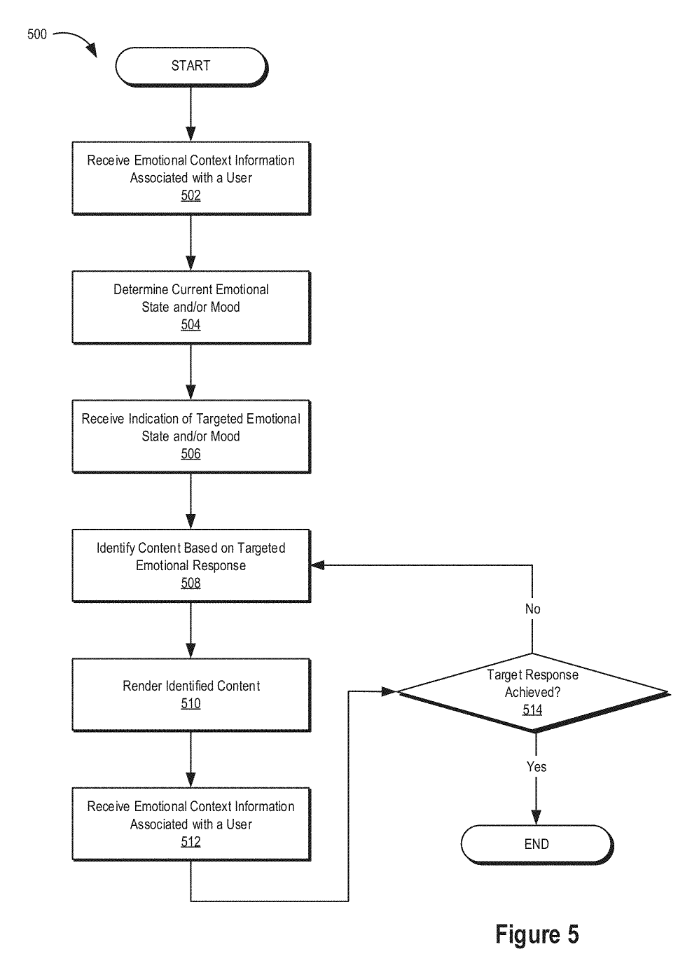

FIG. 5 illustrates an exemplary method 500 for emotional response targeting using contextual information consistent with embodiments of the present disclosure. The illustrated method 500 may be implemented in a variety of ways, including using software, firmware, hardware, and/or any combination thereof. In certain embodiments, various aspects of the method 500 may be implemented by an electronic device associated with a user, a trusted service, and/or a content and/or targeting service.

Various embodiments disclosed herein may use emotional context information associated with content to target and/or evoke a particular emotional response in a user. Emotional context information may be received from one or more emotional context information sensors included in a user device and/or other devices and/or systems associated with the user device at 502. The emotional context information may include, for example, one or more of audio information relating to a user's voice, image and/or video information relating to a user's facial expressions, biometric and/or physiological information, other sensor data, and/or any other relevant information indicative of a user's emotional state and/or mood.

Based on the emotional context information received at 502, a current emotional state and/or mood associated with the user may be inferred and/or otherwise determined at 504. In some embodiments, inferred emotional response information may comprise weighted, scored, and/or otherwise quantified indications of likely user emotional states and/or moods based on the emotional context information.

At 506, an indication of a target emotional state and/or mood may be received. Content may be identified at 508 configured to evoke a target emotional response in a user. That is, at 508, content may be identified which when rendered is likely to cause a user's current emotional state and/or mood determined at 504 to change to the target emotional state and/or mood specified by the indication received at 506.

In some embodiments, the content may be identified at 508 independent of the current emotional state and/or mood of the user determined at 504. For example, the content may be identified at 508 by simply analyzing metadata associated with available content that includes emotional context information to identify metadata matches and/or is similar within a certain degree and/or threshold to emotional context information that is associated with the target emotional state and/or mood specified by the indication received at 506.

In further embodiments, the identification of the content at 508 may be based on the current emotional state and/or mood of the user determined at 504 and the targeted emotional state and/or mood of the user specified by the indication received at 506. For example, content may selected to evoke a transition from a specific first emotional state and/or mood to a targeted emotional state and/or mood (e.g., sad to happy). In this manner, content may be identified at 508 that designed to evoke a particular transition to a target emotional state and/or mood of a user.

The content identified at 508 may be rendered to a user at 510. During and/or following rendering of the content, emotional context information may be received associated with the user at 512. In some embodiments, emotional context information received at 512 may be used to infer and/or otherwise determine the influence of the content rendered at 510 on the emotional state and/or mood of the user. At 514, a determination may be made regarding whether the emotional context information received at 512 is similar within a certain degree and/or threshold to emotional context information that is associated with the target emotional state and/or mood specified by the indication received at 506. If so, an associated indication may be send to an interested entity and the method 500 may proceed to end.

If the emotional context information received at 512 is not similar within a certain degree and/or threshold to emotional context information that is associated with the targeted emotional state and/or mood specified by the indication received at 506, the method 500 may return to 508, where content may be identified based on the emotional context information received at 512. In this manner, content may be iteratively identified at 508 and rendered at 510 until a desired user emotional state and/or mood is achieved.

Embodiments of the disclosed method 500 may be implemented in a variety of contexts. For example, a restaurant may wish to evoke a romantic mood in its patrons. The restaurant may thus wish to play music that is tagged and/or otherwise associated with emotional context information associated with a romantic emotional state. Emotional context information associated with restrauant patrons may be collected (e.g., via cameras, microphones, and/or other emotional context information sensors associated with an environment of the patrons and/or devices associated with the patrons), and the collected information may be used to identify content that when rendered in the restaurant may evoke the desired emotional response. In some circumstances, selected content may be dynamically updated based on feedback from real-time measured emotional context information associated with a target audience.

In another example, a performance event may wish to encourage an audience to feel a particular way (e.g., euphoric). Accordingly, the event may play video content that that is associated with a target emotional state associated with the desired audience feelings. In some circumstances, selected content may be dynamically updated based on real-time measured emotional context information associated with a target audience.

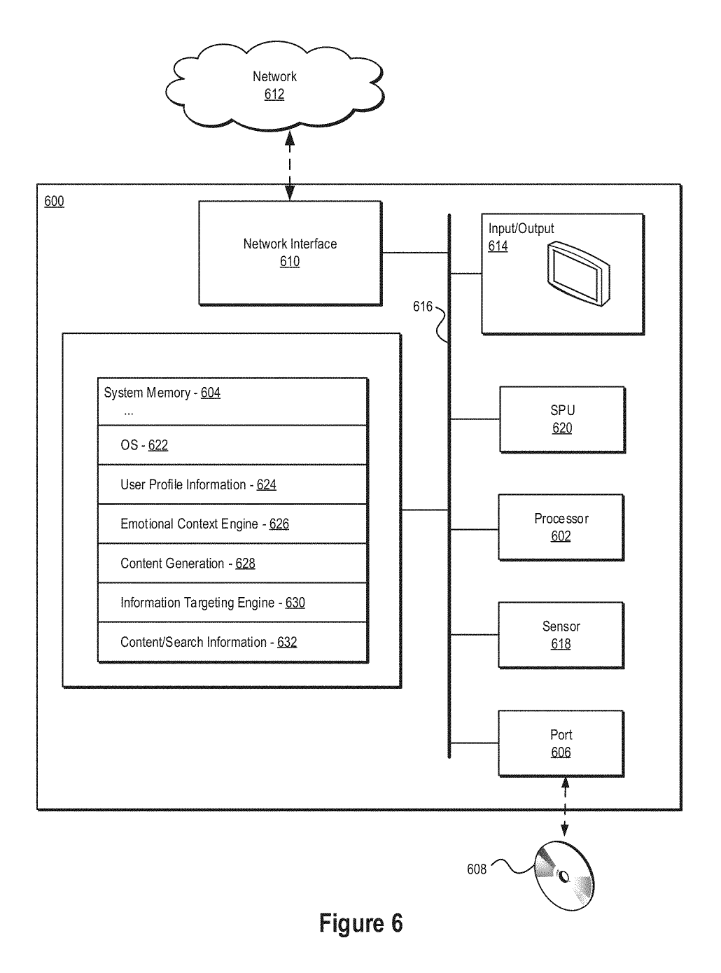

FIG. 6 illustrates an exemplary system 600 that may be used to implement embodiments of the systems and methods of the present disclosure. Certain elements associated with the illustrated exemplary system may be included in a user device, trusted service, an information service such as a content/search service, and/or any other system configured to implement embodiments of the disclosed systems and methods. As illustrated in FIG. 6, the system may include: a processing unit 602; system memory 604, which may include high speed random access memory (“RAM”), non-volatile memory (“ROM”), and/or one or more bulk non-volatile non-transitory computer-readable storage mediums (e.g., a hard disk, flash memory, etc.) for storing programs and other data for use and execution by the processing unit; a port 606 for interfacing with removable memory 608 that may include one or more diskettes, optical storage mediums, and/or other non-transitory computer-readable storage mediums (e.g., flash memory, thumb drives, USB dongles, compact discs, DVDs, etc.); a network interface 610 for communicating with other systems via one or more network connections 612 using one or more communication technologies; a user interface 614 that may include a display and/or one or more input/output devices such as, for example, a touchscreen, a keyboard, a mouse, a track pad, and the like; and one or more busses 616 for communicatively coupling the elements of the system. In certain embodiments, the system 600 may include and/or be associated with one or more sensors 618 configured to collect various information including contextual user information. Such sensors 618 may comprise, without limitation, audio sensors, video and/or image sensors, and/or any other types of emotional context information sensors disclosed herein.

In some embodiments, the system may, alternatively or in addition, include an SPU 620 that is protected from tampering by a user of the system or other entities by utilizing secure physical and/or virtual security techniques. An SPU 620 can help enhance the security of sensitive operations such as personal information management, trusted credential and/or key management, privacy and policy management, and other aspects of the systems and methods disclosed herein. In certain embodiments, the SPU 620 may operate in a logically secure processing domain and be configured to protect and operate on secret information, as described herein. In some embodiments, the SPU 620 may include internal memory storing executable instructions or programs configured to enable the SPU to perform secure operations, as described herein.

The operation of the system 600 may be generally controlled by a processing unit 602 and/or an SPU 620 operating by executing software instructions and programs stored in the system memory 604 (and/or other computer-readable media, such as removable memory). The system memory 604 may store a variety of executable programs or modules for controlling the operation of the system. For example, the system memory may include an operating system (“OS”) 622 that may manage and coordinate, at least in part, system hardware resources and provide for common services for execution of various applications and a trust and privacy management system for implementing trust and privacy management functionality including protection and/or management of personal data through management and/or enforcement of associated policies. The system memory 604 may further include, without limitation, communication software configured to enable in part communication with and by the system; one or more applications; user profile information 624; an emotional context information engine 626 configured to analyze available contextual information, infer one or more user moods and/or emotional states based on the available contextual information, and/or assign one or more weights to the inferred moods and/or emotional states; one or more content generation applications 628 configured to allow a user to generate content using the system 600 (e.g., audio content, video content, image content, etc.); an information targeting engine 630 such as a content/search targeting engine configured to target information to the interests, mood, and/or emotional state of a user; a repository of associated information such as content and/or search information 632; and/or any other information and/or applications configured to implement embodiments of the systems and methods disclosed herein.

The systems and methods disclosed herein are not inherently related to any particular computer, device, service, or other apparatus and may be implemented by a suitable combination of hardware, software, and/or firmware. Software implementations may include one or more computer programs comprising executable code/instructions that, when executed by a processor, may cause the processor to perform a method defined at least in part by the executable instructions. The computer program can be written in any form of programming language, including compiled or interpreted languages, and can be deployed in any form, including as a standalone program or as a module, component, subroutine, or other unit suitable for use in a computing environment. Further, a computer program can be deployed to be executed on one computer or on multiple computers at one site or distributed across multiple sites and interconnected by a communication network. Software embodiments may be implemented as a computer program product that comprises a non-transitory storage medium configured to store computer programs and instructions, that when executed by a processor, are configured to cause the processor to perform a method according to the instructions. In certain embodiments, the non-transitory storage medium may take any form capable of storing processor-readable instructions on a non-transitory storage medium. A non-transitory storage medium may be embodied by a compact disk, digital-video disk, an optical storage medium, flash memory, integrated circuits, or any other non-transitory digital processing apparatus memory device.

Although the foregoing has been described in some detail for purposes of clarity, it will be apparent that certain changes and modifications may be made without departing from the principles thereof. It should be noted that there are many alternative ways of implementing both the systems and methods described herein. Accordingly, the present embodiments are to be considered as illustrative and not restrictive, and the invention is not to be limited to the details given herein, but may be modified within the scope and equivalents of the appended claims.

What is claimed is:

1. A method performed targeting content based on a desired emotional response performed by a system comprising a processor and a non-transitory computer-readable medium storing instructions that, when executed by the processor, cause the system to perform the method, the method comprising:
receiving first emotional context information associated with a target user;
determining a first emotional state of the user based on the first emotional context information;
receiving an indication of a target emotional state, the target emotional state being different than the first emotional state;
identifying content based on the target emotional state; and
rendering the identified content to the target user.

2. The method of claim 1, wherein the method further comprises:
receiving, after rendering the identified content to the target user, second emotional context information associated with the target user;
determining a second emotional state of the user based on the emotional context information; and
comparing the second emotional state with the target emotional state.

3. The method of claim 2, wherein comparing the second emotional state with the target emotional state comprises determining that the second emotional state is within a threshold degree of similarity with the target emotional state.

4. The method of claim 3, wherein the method further comprises:
generating an indication that target emotional state was achieved.

5. The method of claim 1, wherein the method further comprises:
determining a targeted emotional response based on the first emotional state and the target emotional state, and
wherein identifying the content is further based on the target emotional response.

6. The method of claim 1, wherein the first emotional context information comprises information obtained by one or more emotional context information sensors.

7. The method of claim 6, wherein the one or more emotional context information sensors comprise one or more sensors included in an environment of the target user.

8. The method of claim 6, wherein the one or more emotional context information sensors comprise one or more sensors associated with a device of the target user.

9. The method of claim 6, wherein the one or more emotional context information sensors comprise at least one of a microphone, an image camera, a video camera, a temperature sensor, a heart rate sensor, a blood pressure sensor, a temperature sensor, a perspiration level sensor, a pressure sensor, and a light sensor.

10. The method of claim 6, wherein the first emotional context information comprises at least one of a tone of the target user's voice, a decibel level of the target user's voice, recognized speech content of the target user's voice, facial expression recognition information, iris dilation information, heart rate information, blood pressure information, temperature information, perspiration information, user interface pressure sensor data, light sensor data, and device usage information.
